(19) United States
(12) Patent Application Publication
(10) Pub. No.: US 2011/0201973 A1
(43) Pub. Date: Aug. 18, 2011

(54) ULTRASOUND COMPATIBLE RADIOFREQUENCY ABLATION ELECTRODE

(75) Inventors: Douglas N. STEPHENS, Davis, CA (US); Alan DE LA RAMA, Cerritos, CA (US); Peter C. CHEN, Irvine, CA (US)

(73) Assignee: ST. JUDE MEDICAL, INC., St. Paul, MN (US)

(21) Appl. No.: 13/019,690

(22) Filed: Feb. 2, 2011

Related U.S. Application Data
(60) Provisional application No. 61/305,693, filed on Feb. 18, 2010.

(51) Int. Cl. A61N 7/00

(52) U.S. Cl. 601/2

(57) ABSTRACT

Embodiments of the present invention are directed to an ultrasound compatible ablation electrode for use in ultrasound imaging guidance of ablation therapy using RF or the like. In one embodiment, an ultrasound compatible ablation catheter comprises a catheter body having a distal end and an ultrasonic transducer directing ultrasonic beams for imaging a target; and an ablation electrode connected to the catheter body, the ablation electrode having a plastic shell and a metallic coating on the plastic shell which are disposed in a path of the ultrasonic beams of the ultrasonic transducer between the ultrasonic transducer and the target, the metallic coating of the ablation electrode to be energized for ablation.

This application claims the benefit of U.S. Provisional Patent Application No. 61/305,693, filed Feb. 18, 2010, the entire disclosure of which is incorporated herein by reference.

### BACKGROUND OF THE INVENTION

The present invention relates generally to ablation devices and, more specifically, to an ultrasound compatible radiofrequency (RF) ablation electrode.

Catheters are flexible, tubular devices that are widely used by physicians performing medical procedures to gain access into interior regions of the body. For example, ablation catheters are sometimes used to perform ablation procedures to treat certain conditions of a patient. A patient experiencing arrhythmia, for example, may benefit from ablation to prevent irregular heart beats caused by arrhythmogenic electrical signals generated in cardiac tissues. By ablating or altering cardiac tissues that generate such unintended electrical signals the irregular heart beats may be stopped. Ablation catheters are known, and may include one or more ablation electrodes supplying RF (radiofrequency) energy to targeted tissue. With the aid of sensing and mapping tools that are also known, an electrophysiologist can determine a region of tissue in the body, such as cardiac tissue, that may benefit from ablation. One technique utilizes ultrasound imaging guidance for RF ablation therapy. See, e.g., U.S. Patent Application Publication No. 2007/0021744.

### BRIEF SUMMARY OF THE INVENTION

Embodiments of the present invention are directed to an ultrasound compatible ablation electrode for use in ultrasound imaging guidance of ablation therapy using RF or the like. The ablation electrode incorporates a plastic body coated with a thin metal film that provides electrical contact for RF ablation or the like and at the same time allows ultrasound to penetrate easily therethrough without substantial artifacts in the resulting image. The unique structure of the ablation electrode facilitates the proper functioning of two otherwise incompatible modalities that are RF tissue ablation and ultrasound imaging. As a result, an operator can use the ablation electrode to ultrasonically visualize the tissue to ablate and to ablate the tissue simultaneously in real time. Some of the advantages of this approach include a more precise placement of the catheter in or on the tissue to be ablated, improved ultrasound visualization of the ablation process including clot formation and tissue changes during and after ablation, and better decision making on the movement of the catheter if a linear or pattern ablation is to be made. The ablation electrode desirably has a dome or curved shape and includes a fluid cavity with one or more cooling fluid entry ports and one or more cooling fluid exit ports for an irrigated catheter.

In accordance with an aspect of the present invention, an ultrasound compatible ablation catheter comprises a catheter body having a distal end and an ultrasonic transducer directing ultrasonic beams for imaging a target; and an ablation electrode connected to the catheter body, the ablation electrode having a plastic shell and a metallic coating on the plastic shell which are disposed in a path of the ultrasonic beams of the ultrasonic transducer between the ultrasonic transducer and the target, the metallic coating of the ablation electrode to be energized for ablation.

In specific embodiments, the plastic shell has an acoustic impedance magnitude which is in a range of 1500×10³ to 1750×10³ Rayls (kg/m²s) at a temperature of 37° C. The metallic coating is substantially thinner than the plastic shell. The thickness of the plastic shell is preferably at least about 10 times the thickness of the metallic coating. The plastic shell has a thickness of at most about 500 microns and the metallic coating has a thickness of at most about 20 microns. The plastic shell comprises TPX® (polymethylpentene). The ablation region further comprises an electrical barrier layer on an exterior surface of the metallic coating, the barrier layer being substantially thinner than the metallic coating. The catheter body and the ablation electrode form a fluid cavity to store a fluid through which the ultrasonic beams of the ultrasonic transducer are transmitted across the ablation electrode to the target. The plastic shell has an acoustic impedance which is substantially equal to an acoustic impedance of the fluid. The catheter further comprises at least one fluid entry port for the fluid cavity, and at least one fluid exit port for the fluid cavity.

In specific embodiments, the ablation electrode comprises an ablation tip disposed near the distal end. The ablation tip is dome-shaped to provide a rounded ablation surface on the metallic coating. The ablation electrode has one of an uneven surface or a faceted surface to scatter reflective energy of the ultrasonic beams passing therethrough between the ultrasonic transducer and the target. The ultrasonic transducer is disposed on the distal end of the catheter body and comprises an array for forward looking imaging.

In some embodiments, the ultrasonic transducer and the ablation electrode are disposed on the catheter body, and the ultrasonic transducer comprises an array for side looking imaging. The ultrasonic transducer and the ablation electrode are disposed on opposite sides with respect to a longitudinal axis of the catheter body.

In specific embodiments, the catheter further comprises a control unit which controls an ultrasound generator to supply ultrasound energy to the ultrasonic transducer, an ultrasound receiver to accept echo signals, and an RF energy source to supply RF energy to the metallic coating of the ablation electrode, for ultrasound imaging and RF ablation simultaneously. The ablation electrode is constructed of materials and thicknesses to produce an absorption loss of less than about 50% of ultrasonic beam energy of the ultrasonic beams of the ultrasonic transducer for imaging the target.

In accordance with another aspect of the invention, an ultrasound compatible ablation catheter comprises a catheter body having a distal end and an ultrasonic transducer directing ultrasonic beams for imaging a target; and an ablation electrode connected to the catheter body, the ablation electrode having a plastic shell and a metallic coating on the plastic shell which are disposed in a path of the ultrasonic beams of the ultrasonic transducer between the ultrasonic transducer and the target, the metallic coating of the ablation electrode to be energized for ablation. The catheter body and the ablation electrode form a fluid cavity to contain a fluid through which the ultrasonic beams of the ultrasonic transducer are transmitted across the ablation electrode to the target. The plastic shell has an acoustic impedance magnitude which is in a range of 1500×10³ to 1750×10³ Rayls (kg/m²s) at a temperature of 37° C. The metallic coating is substantially thinner than the plastic shell.

In accordance with another aspect of the invention, an ultrasound compatible ablation catheter comprises a catheter body having a distal end and an ultrasonic transducer directing ultrasonic beams for imaging a target; and an ablation electrode connected to the catheter body, the ablation electrode having a plastic shell and a metallic coating on the plastic shell which are disposed in a path of the ultrasonic beams of the ultrasonic transducer between the ultrasonic transducer and the target, the metallic coating of the ablation electrode to be energized for ablation. The catheter body and the ablation electrode form a fluid cavity to contain a fluid through which the ultrasonic beams of the ultrasonic transducer are transmitted across the ablation electrode to the target. The plastic shell has an acoustic impedance which is substantially equal to an acoustic impedance of the fluid. The metallic coating is substantially thinner than the plastic shell.

These and other features and advantages of the present invention will become apparent to those of ordinary skill in the art in view of the following detailed description of the specific embodiments.

### BRIEF DESCRIPTION OF THE DRAWINGS

FIG. 1 shows an ultrasound compatible ablation tip for a catheter according to an embodiment of the present invention.

FIG. 2 is a schematic illustration of an ultrasound transducer in the form of a microlinear array disposed near the catheter tip of FIG. 1.

FIG. 3 shows a number of alternative shapes for the ablation tip.

FIG. 4 is a block diagram illustrating the electrical functions of an ablation and imaging system.

FIG. 5 is a plot showing that an ECG triggered strobing of the RF generator may permit ultrasonic (US) tracking of tissue changes during the heating of RFA.

FIG. 6 is a schematic illustration of another ultrasound compatible ablation tip for a catheter.

FIG. 7 shows a faceted surface for the ablation tip.

FIG. 8 shows an ultrasound compatible ablation tip for a catheter according to another embodiment of the invention.

FIG. 9a is a schematic illustration of an ultrasound transducer in the form of a microlinear array disposed in the body of the catheter tip and parallel with the longitudinal dimension of the catheter.

FIG. 9b is a schematic illustration of an ultrasound transducer in the form of a microlinear array disposed in the body of the catheter tip and perpendicular with the longitudinal dimension of the catheter.

FIG. 9c is a schematic illustration of an ultrasound transducer in the form of a microlinear array disposed in the body of the catheter tip with both parallel and perpendicular features with respect to the longitudinal dimension of the catheter.

FIG. 10 is a side view of a catheter schematically illustrating an ultrasound compatible ablation member for a catheter with a premounted side viewing ultrasound array.

FIG. 11 is a perspective view illustrating different ultrasound compatible ablation members.

FIG. 12 is a side sectional view of the side viewing catheter of FIG. 10.

FIG. 13 shows a set of sectional views of the side viewing catheter of FIG. 12 illustrating different bi-directional steering configurations.

FIGS. 14a and 14b show different arrangements of hole shapes and positions in the ultrasound compatible ablation member to permit irrigation fluid flow.

### DETAILED DESCRIPTION OF THE INVENTION

In the following detailed description of the invention, reference is made to the accompanying drawings which form a part of the disclosure, and in which are shown by way of illustration, and not of limitation, exemplary embodiments by which the invention may be practiced. In the drawings, like numerals describe substantially similar components throughout the several views. Further, it should be noted that while the detailed description provides various exemplary embodiments, as described below and as illustrated in the drawings, the present invention is not limited to the embodiments described and illustrated herein, but can extend to other embodiments, as would be known or as would become known to those skilled in the art. Reference in the specification to “one embodiment,” “this embodiment,” or “these embodiments” means that a particular feature, structure, or characteristic described in connection with the embodiment is included in at least one embodiment of the invention, and the appearances of these phrases in various places in the specification are not necessarily all referring to the same embodiment. Additionally, in the following detailed description, numerous specific details are set forth in order to produce a thorough understanding of the present invention. However, it will be apparent to one of ordinary skill in the art that these specific details may not all be needed to practice the present invention. In other circumstances, well-known structures, materials, circuits, processes and interfaces have not been described in detail, and/or may be illustrated in block diagram form, so as to not unnecessarily obscure the present invention.

In the following description, relative orientation and placement terminology, such as the terms horizontal, vertical, left, right, top and bottom, is used. It will be appreciated that these terms refer to relative directions and placement in a two dimensional layout with respect to a given orientation of the layout. For a different orientation of the layout, different relative orientation and placement terms may be used to describe the same objects or operations.

Exemplary embodiments of the invention, as will be described in greater detail below, provide apparatuses and methods for ultrasound imaging guidance of RF ablation therapy using an ultrasound compatible RF ablation electrode.

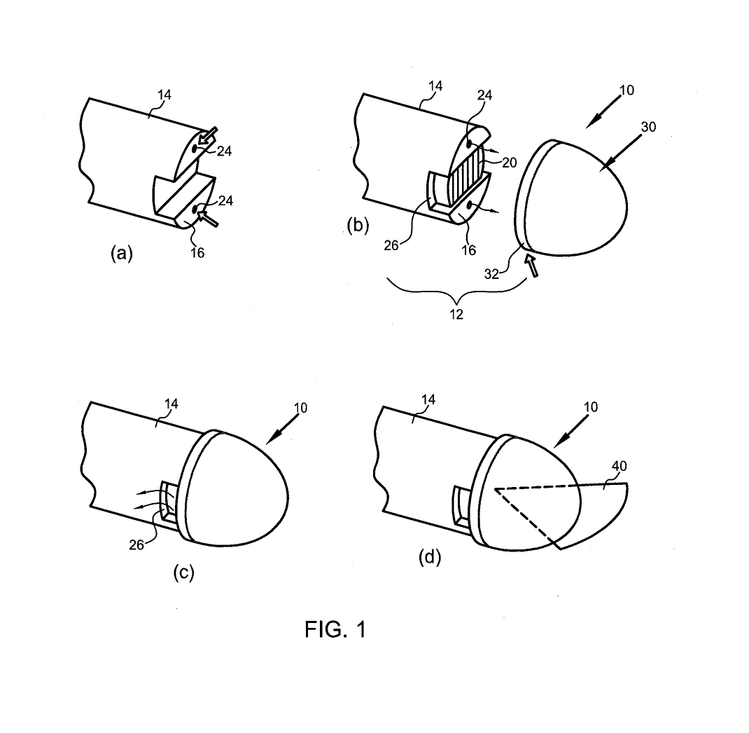

FIG. 1 shows an ultrasound compatible ablation electrode 10 for a catheter 12 according to an embodiment of the present invention. The ablation electrode 10 is connected to the catheter shaft or body 14 at or near a distal end 16 thereof. As seen in FIG. 1b, an ultrasound transducer 20 is provided at the distal end 16 of the catheter shaft 14 to direct ultrasonic beams for imaging a target. The shaft distal end 16 includes one or more irrigation fluid entry ports 24 for directing irrigation fluid into the interior of the ablation electrode 10. In this embodiment, the irrigation fluid exits via one or more irrigation fluid exit ports 26 provided at the side edges of the distal end 16 of the catheter shaft 14. Alternative or additionally, one or more fluid exit ports may be provided on the ablation electrode 10 (typically at or near the apex). The distal end 16 of the catheter body 14 and the ablation electrode 10 form a fluid cavity to store a fluid through which the ultrasonic beams of the ultrasonic transducer 20 are transmitted across the ablation electrode 10 to the target.

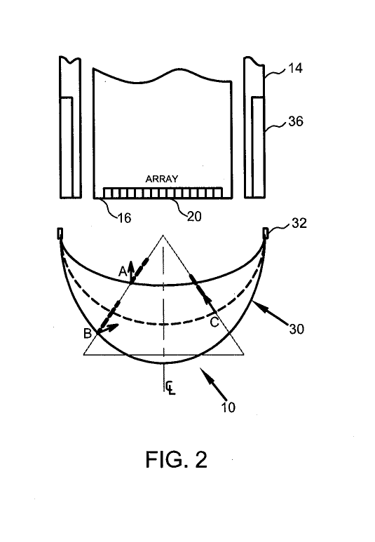

The ablation electrode 10 has a dome shape which may be generally spherical or elliptical, and is made of a plastic shell coated with a thin electrically conductive metal layer 30 on the outer surface. The metal layer 30 provides a rounded or dome-shaped ablation surface to provide a smooth atraumatic exterior and as a means of reflecting undesirable ultrasound echoes (see “A” and “B” in FIG. 2) away from a direct return path (see “C” in FIG. 2) to the ultrasound transducer. The plastic shell and the metallic coating 30 of the ablation electrode 10 are disposed in the path of the ultrasonic beams between the ultrasonic transducer 20 and the imaging target. The metallic coating 30 is to be energized for ablation by an energy source (e.g., electrical, RF, etc.). A metal contact 32 around the edge of the ablation electrode 10 provides electrical contact between the metal layer 30 of the ablation electrode 10 and an RF electrode 36 around the ultrasound transducer 20 on the catheter shaft 14 (see FIG. 2). The metal contact 32 may be a continuous band wrapped around the entire circumference or one or more discrete contact segments. The metal contact 32 may be eliminated in some embodiments. The RF electrode 36 may be a metal tube-like electrode or a plurality of discrete electrode segments. In this embodiment, the ablation electrode 36 is unipolar, and the other electrode is typically underneath the patient and contacts the posterior chest wall of the patient, for example.

The ultrasound transducer 20 may be configured in the form of an array. FIG. 1d schematically illustrates an image plane 40 of the ultrasound transducer 20 for phased array sector imaging. In specific embodiments, the transducer 20 is a forward looking microlinear array having, for instance, a 24-element, 14 MHz phased array mounted at the distal end 16 of the catheter body 14 for high definition, high-frame rate, forward looking imaging. The microlinear array 20 is a piezoceramic or a capacitive micromachined ultrasonic transducer (CMUT) array in which the flex circuit itself and a single thin parylene layer serve as the effective acoustic matching layers. The piezoceramic array is designed as a 2-2 composite structure using a high-dielectric ceramic. The core microlinear array design is based on a stacked structure in which one piezoceramic “layer” in the 2-2 lead zirconate titanate (PZT) composite defines a single element before bonding the array to the flex circuit. The composite is about 112 μm thick with 50-μm wide piezoceramic “stacked” elements and 15-μm epoxy-filled kerfs. The front-side materials are a 25-μm thick polyimide flex circuit with 4.5-μm metal traces that make an electrical contact by compression through the bonding epoxy with the 2-2 composite structure and a parylene layer at 10 μm of thickness to serve as an insulating outer layer that prevents the flex circuit outside (ground shield) metal from touching biological tissue. The backing is a cast-on electrically conductive reference electrode side of approximately 1 mm in thickness. The microlinear array flex circuit assembly places the active signal wiring on the inside flex bend where solder connections to the internal coaxial cabling are made along with the ground wire connecting the piezoceramic grounded back-side connection. The microlinear array is described in Douglas N. Stephens et al., Experimental Studies with a 9F Forward-Looking Intracardiac Imaging and Ablation Catheter, J Ultrasound Med 2009; 28:207-215 (2009); Douglas N. Stephens et al., Multifunctional Catheters Combining Intracardiac Ultrasound Imaging and Electrophysiology Sensing, IEEE Transactions on Ultrasonics, Ferroelectrics, and Frequency Control, Vol. 55, No. 7, 1570-1581 (July 2008); and Amin Nikoozadeh et al., Forward-Looking Intracardiac Ultrasound Imaging Using a 1-D CMUT Array Integrated with Custom Front-End Electronics, IEEE Transactions on Ultrasonics, Ferroelectrics, and Frequency Control, Vol. 55, No. 12, 2651-2660 (December 2008), the entire disclosures of which are incorporated herein by reference.

FIG. 2 is a schematic illustration of an ultrasound transducer 20 in the form of a microlinear array disposed near the catheter tip 10. The catheter shaft 14 has one or more RF electrodes 36 at its distal end which make contact with the catheter tip 10 via the one or more metal contacts 32 on the periphery of the distal end of the shaft 14. The triangle represents the region of beam forming used by the array 20. The three arrows from points A, B, and C show the directions of specular ultrasound reflections from the metal film 30. Points A and B are just representative example points with beam reflections in the cases of low radius and high radius curvatures respectively. The point C on the dashed curve is a representative point on a perfectly round (i.e., spherical) surface. A and B are reflective echo paths which, due to special domed shapes, show that echoes can be reflected away from a direct return path to the transducer array 20. C, on the other hand, shows the undesirable reflected echo which can return directly to the array with an ordinary circular shaped electrode.

Generally speaking, the ultrasound imaging pathway must be free of obstructions to permit ultrasound beam energy to penetrate to tissue depths so that the echo reflections can be visualized. Previous RF ablation tips are made of relatively thick metal with relatively broad contact areas to permit good distribution of RF energy into the tissue to be ablated, but they also obstruct ultrasound beam energy. In contrast, ultrasound, even at the higher common frequencies around 10 to 30 MHz, can penetrate easily a thin plastic such as TPX® (polymethylpentene, TPX is a trademark of Mitsui Chemicals, Inc.) without many artifacts in the resulting image. The addition of a thin metal film on the thin plastic ablation electrode is permissible, as long as the metal film is not too thick. Heretofore in RF ablation methodologies, a thin conductive metal layer would generally not be used due to the potential for heating at the ablation electrode and for reasons of mechanical strength. The present ablation electrode 10 allows for both cooling with irrigation fluid and strength in the use of a dome-like contact. In this design, a reasonable compromise can be struck between the needs of both ultrasound and ablation modalities.

The materials and dimensions of the ablation electrode 10 are chosen such that ultrasound from the transducer 20 to be used for image guidance and procedural feedback can penetrate the ablation electrode 10 without substantial distortion, so as to permit reasonably good imaging results of the tissue just beyond the ablation electrode 10. For instance, the ultrasound reflected intensity ratio with respect to the transmitted intensity is preferably less than about 0.01, more preferably less than about 0.0001, and most preferably less than about 10⁻⁶. The dome-like structure of the ablation electrode 10 provides a desirable broad surface at the electrode of a therapeutic ablation catheter which is metallized for the purpose of supplying a high power radiofrequency electrical signal to tissues of the body intended for thermal ablative therapy. The catheter includes irrigation fluid cooling of the ablation electrode 10 which is desirable, both as a way of avoiding tissue surface contact “charring” from the ablation and as a way of cooling the thin metal surface from heating beyond the a temperature suitable for the plastic material upon which the metal is supported. The moving water also helps prevent any air bubbles from forming and adhering to the inside of the tip.

The plastic shell of rounded, or faceted, shape is relatively thin and the metal coating is even thinner. The metallic coating is substantially thinner than the plastic shell (i.e., at least several times thinner). For example, the thickness of the plastic shell is preferably at least about 10 times the thickness of the metallic coating. In specific embodiments, the plastic shell has a thickness of equal to or less than about 500 microns (e.g., in the range of about 20 to 500 microns) and the metallic coating has a thickness of equal to or less than about 20 microns (e.g., in the range of several microns to possibly slightly more than 20 microns). The thicknesses discussed here are generally to be designed in an inverse relation with the frequency of ultrasound used. To make use of 10 MHz ultrasound imaging, for example, the plastic shell could be about 75 microns and the metallic coating a total thickness of about 3 microns.

For efficient transmission of the ultrasonic beams through the ablation electrode 10, the plastic shell has an acoustic impedance which preferably is substantially equal to an acoustic impedance of the fluid inside the fluid cavity formed by the distal end 16 of the catheter body 14 and the ablation electrode 10. The fluid is typically water or saline which produces low absorption loss of high frequency ultrasound in the range of about 5-30 MHz used for imaging. In use, the catheter 12 is typically inside a blood vessel with blood flowing therethrough. The acoustic impedance of blood is reasonably close to that of water or saline. The acoustic impedance magnitude of blood at 37° C. is the product of its acoustic velocity (1590 m/s) and density (1.06 g/cm³) (see, e.g., F. A. Duck, The Physical Properties of Tissue: A Comprehensive Reference Book, San Diego, Calif., Academic Press, Inc., 1990) or 1680×10³ Rayls (kg/m²s). The acoustic impedance magnitude of cardiac tissue is very close to this impedance as well. In specific embodiments, the plastic shell has an acoustic impedance magnitude which is substantially equal to about 1680×10³ Rayls (kg/m²s) at a temperature of about 37° C. The metallic coating 30 has a different acoustic impedance magnitude, but its effect on the absorption loss is kept relatively insignificant due to its small thickness.

The ablation electrode 10 is based upon several design elements that work well together. These design elements include the use of a plastic material (e.g., TPX) which has a low absorption loss at even high frequency ultrasound (e.g., about 10 MHz), the use of a high conductivity metal layers (e.g., platinum-iridium, or chrome/gold, or titanium, nickel, gold, etc.) which provides a good, large surface area for a low resistive contact to body tissues, the use of a dome-like electrode shape for the ablation electrode 10 which is strong (to prevent crushing upon contact) and provides a good contact surface that is not necessarily position dependent (for ease in contact with tissues), and the use of open irrigation fluid flow which is supplied by the catheter fluid channels to both cool the ablation electrode and help maintain its general dome-like shape via fluid flow pressure. The absorption loss depends on the acoustic impedance values of the ablation electrode as well as the material properties and thicknesses of the plastic shell and metallic coating of the ablation electrode. The ablation electrode 10 is constructed of materials and thicknesses to produce an absorption loss of preferably less than about 10% of the ultrasonic beam energy of the ultrasonic beams of the ultrasonic transducer 20 for imaging the target, more preferably less than about 1%, and most preferably less than about 0.1%.

In alternative embodiments, various aspects of the ablation electrode can be adjusted to optimize the design for specific operating conditions or environments. One example is the construction of the plastic shell, which may include TPX variants for the material, the thickness of the shell, the ultrasound characteristics, etc. The shell may be machined from a block or injection molded. Another example is the construction of the metal coating (material, thickness, ultrasound characteristics, etc.). Platinum-iridium is a standard material used in RF ablation devices, and this metal can potentially be sputtered onto the surface of the plastic shell. Gold can be sputtered as well. In any sputtering process in which a plastic is used as the substrate material, a “seed layer” metal is typically used which promotes good adhesion to the plastic substrate. Yet another example is the shape of the dome-like structure for imaging purposes and for mechanical strength reasons.

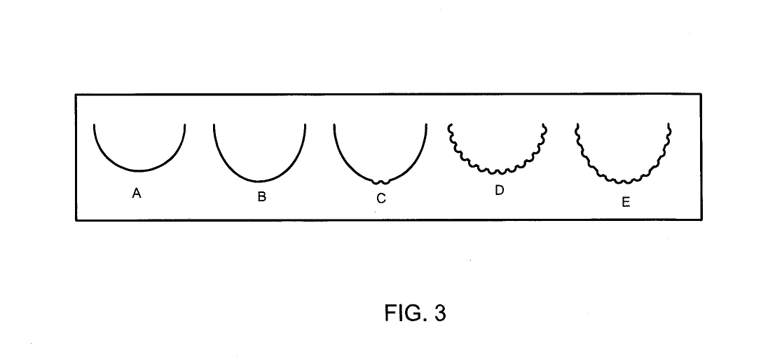

FIG. 3 shows a number of alternative cross-sectional shapes for the ablation electrode 10. FIG. 3A illustrates a spherical shape. FIG. 3B shows an elongated shape that may be generally elliptical. FIG. 3C shows an undulating region with surface features at the distal apex region of the ablation surface which may represent a surface roughness and are configured to reduce or avoid undesired specular reflections that can contribute to image artifacts. The surface features may be dimples, facets, or the like. The specular reflections are typically highest in the apex region of the ablation electrode. The uneven surface of the ablation electrode will scatter reflective energy of the ultrasonic beams passing therethrough between the ultrasonic transducer and the target. The surface features are generally uniform over the entire ablation tips in FIGS. 3D and 3E. The small bar in FIG. 3D shows the ultrasound wavelength at 10 MHz, which is in these designs smaller that the individual surface features. In other embodiments, the surface of the ablation electrode can be made into a plate-like structure (similar to a radar dome).

The surface features of the ablation electrode may be machined or molded. Another method of creating the surface features is by heat treating the plastic shell of the ablation electrode to a temperature slightly below melting so that the plastic shell starts to distort into an irregular shape. The metal layer is formed on the plastic shell with the surface irregularities after the heat treating process. Yet another way to create the surface features is to provide an ablation electrode that is sufficiently flexible such that when the ablation electrode is pressed against tissue to be ablated, the ablation electrode undergoes sufficient flexure or deformation so as to reduce or avoid undesired specular reflections of the imaging ultrasonic beams passing through the ablation electrode.

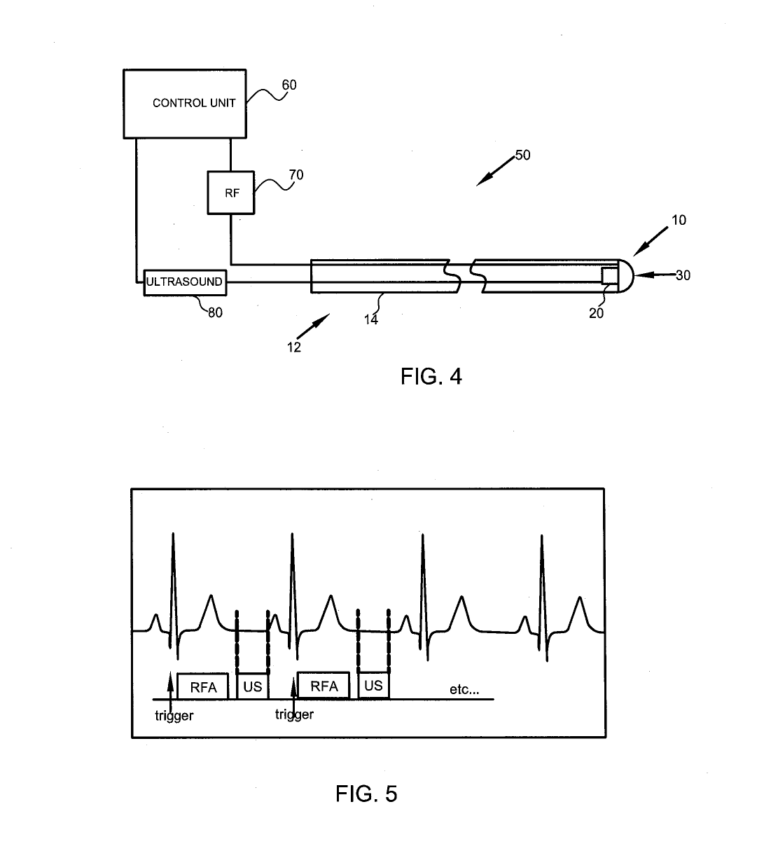

FIG. 4 is a block diagram of the electrical functions of an ablation and imaging system 50 illustrating a control unit 60 which controls an ultrasound generator 80 and an RF energy source 70 to supply ultrasound energy to the ultrasound transducer 20 to transmit ultrasonic beams and to supply RF energy to the metallic coating 30 of the ablation electrode 10, for ultrasound imaging and RF ablation simultaneously. In this way, the operator of the ablation catheter 12 with the ultrasound compatible ablation tip 10 can observe the imaging results at or near the ablation target in real time for image guidance and procedural feedback and perform ablation using the ablation electrode 10 in an integrated manner with the imaging, using a single ablation/imaging catheter that has a broad ablation surface provided by the metallic coating 30 on the dome-shaped ablation electrode 10. For the best ultrasound imaging, the imaging (or ultrasound data collection) would likely need to occur at times when the RF ablation generator is inactive. This is illustrated in FIG. 5 which shows that an ECG triggered strobing of the RF generator may permit ultrasonic (US) tracking of tissue changes in the periods of time between active heating of radio frequency ablation.

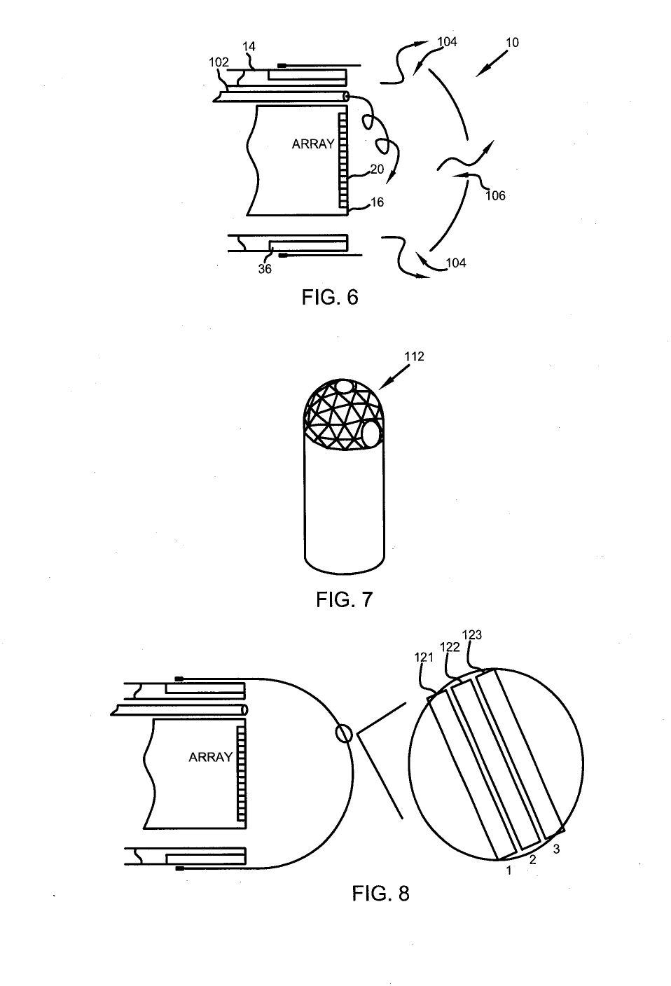

FIG. 6 is a schematic illustration of another ultrasound compatible ablation tip for a catheter. In the MicroLinear (ML) imaging catheter, the RF “electrodes” 36 here are actually one electrode (or they could be two differential electrodes in the embodiment where a split electrode is used). A saline or similar water-like fluid from an irrigation fluid line 102 is used to irrigate the ablation tip 10 of the catheter. The arrows simply show the general pathways for circulation within the tip 10. The saline serves several functions: a) cooling of the tip during ablation, b) removal of air bubble formation if any which may form during the ablation heating, c) transmission medium for the ultrasound which is both produced and received by the array elements of the transducer 20. The holes in the tip 10 are placed so that two or three or more side holes 104 allow for easy exit of the irrigation fluid (and any air bubbles) and allow for blood mixing (which is not required for function but is nonetheless good for saline and blood mixing to keep the catheter tip region cool during ablation). The tip hole 106 is placed so that an ultrasound beam can be used to cross the tip boundary into the tissue in this area which permits a) a relatively unobstructed ultrasound path, and b) tissue surface cooling with the flushing saline. The tip hole 106 is not large (approximately 1 mm in diameter, or simply larger than the −3 dB width of the ultrasound transmission beam using either all or part of the ultrasound array elements to form this beam). The hole dimension is not critical to the operation of the ultrasound compatible ablation tip device, but simply permits a preferred design feature. It is desired not to make the tip hole 106 too large, since a large hole will decrease the possible ablation surface exposure for efficient ablation heating.

FIG. 7 shows a faceted surface 112 with multiple plates or facets for the ablation tip. The faceted surface 112 permits an improved way, perhaps the best way, of scattering any (very small) reflected ultrasound energy of the transmit beam from the transducer array. Any of this very small amount of ultrasound energy which is reflected (since perfect ultrasound transmission through any material is simply not possible) is reflected in such a way as to prevent the coherent summation of this energy at the receiving aperture of the ultrasound array. The facets can be small, or about one half of an ultrasound wavelength (which for 10 MHz ultrasound in blood is about 150 micrometers). The facets are not required to be this small, but many small flat facets will aid in the reduction of a coherently large echo received by the array.

FIG. 8 shows an ultrasound compatible ablation tip for a catheter according to another embodiment of the invention. This ablation tip has three layers instead of two. The first layer 121 is the ultrasound compatible plastic material which has an acoustic impedance close to that of blood, the second layer 122 is the RF ablation electrode metal, and the third layer 123 is an insulation or barrier layer which is very, very thin. The barrier layer 123 is substantially thinner than the metallic layer 122 (e.g., at least about two orders of magnitude thinner). As an example, a barrier layer in the thickness range of approximately 5 to 15 nanometers (0.005 to 0.015 micrometers) with a relative dielectric constant of 3.5 may permit an RF electrical impedance of only about 10 Ohms and thus be used as a coating which can be used in several beneficial ways. The coating can offer increased isolation (at 60 Hz) to protect the patient from electrical shock hazard. The coating could also potentially be used to help in the optimization of the acoustic impedance of the entire layer. This outside coating could be thicker than the nominal dimensions mentioned above if it were also only covering the metal electrode in selected regions, and not in others. This pattern could permit optimal RF ablation modes to heat more of one region of the ablation tip as opposed to others.

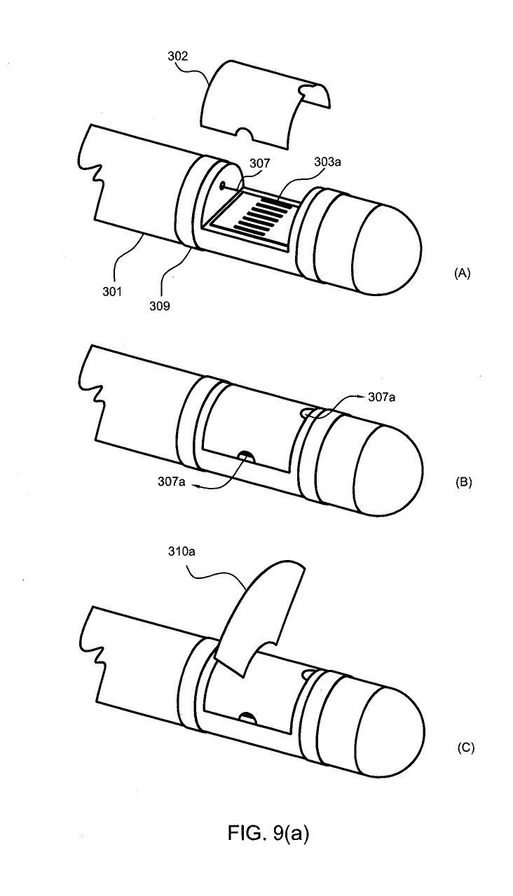

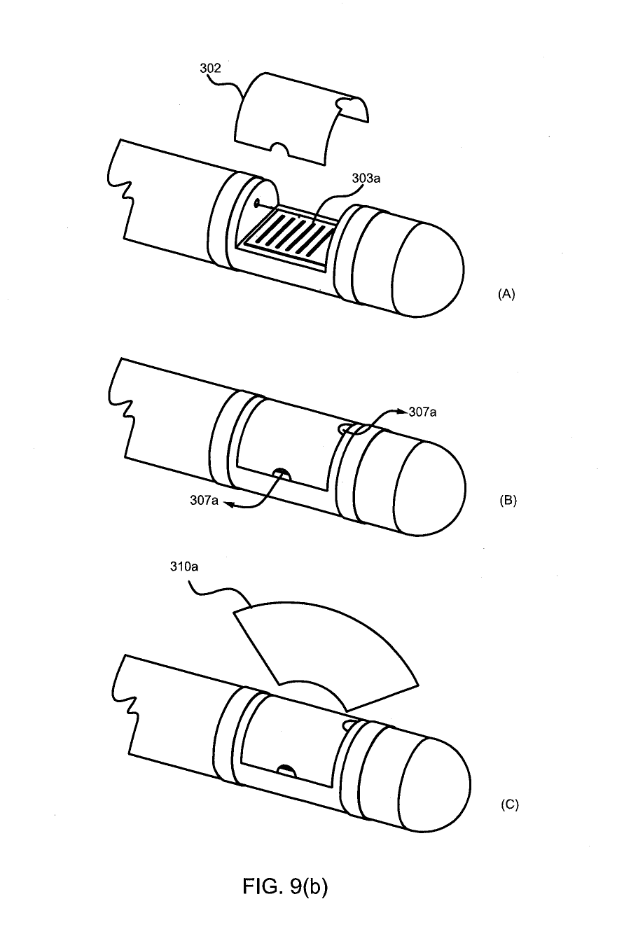

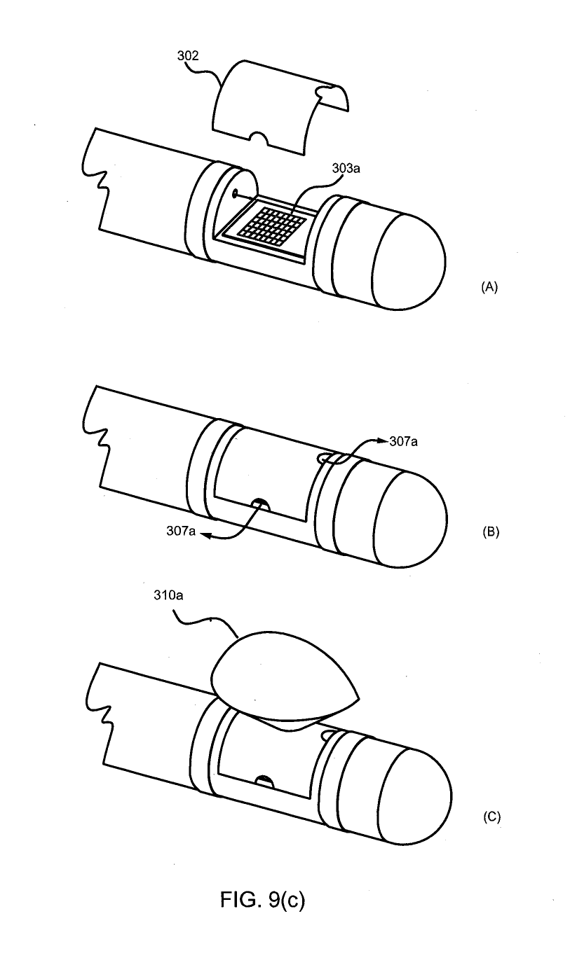

FIG. 9a is a schematic illustration of an ultrasound transducer in the form of a microlinear array disposed in the body of the catheter tip and parallel with the longitudinal dimension of the catheter. A transducer array 203a (one-dimensional array) has elements aligned in parallel with the longitudinal dimension of the catheter which permits an acoustic image plane 210a to exist as a plane at right angle with respect to the catheter shaft. The catheter 201 may be equipped with EP (electrophysiological) electrodes 209 which may be arranged on either side of the imaging array 203a and special metal coated ablation portion arranged as a cylindrical shell portion 202. Water irrigation inflow 207 may be produced which is used both for an ultrasound conduction medium in the region between the array 203a and the metal coated ablation portion 202, and as a coolant for the ablation surface which is the outer metallized surface of the ablation portion 202 in a manner similar to that described for the distal ablation tip of FIGS. 1 and 2. The metal coated ablation portion 202 or the catheter body 201 is equipped with exit ports which allow the irrigation fluid outflow 207a to exit the catheter in a manner similar to that described above for the distal ablation tip.

FIG. 9b is a schematic illustration of an ultrasound transducer in the form of a microlinear array disposed in the body of the catheter tip and perpendicular with the longitudinal dimension of the catheter. The difference in FIG. 9b with respect to FIG. 9a is that the transducer array elements 203b (one-dimensional array) are oriented at 90 degrees to the longitudinal dimension of the catheter, thus producing an image plane 210b in alignment with the longitudinal dimension of the catheter shaft.

FIG. 9c is a schematic illustration of an ultrasound transducer in the form of a microlinear array 203c (two-dimensional array) disposed in the body of the catheter tip with both parallel and perpendicular features with respect to the longitudinal dimension of the catheter. The microlinear array 203c produces a volumetric beam 210c.

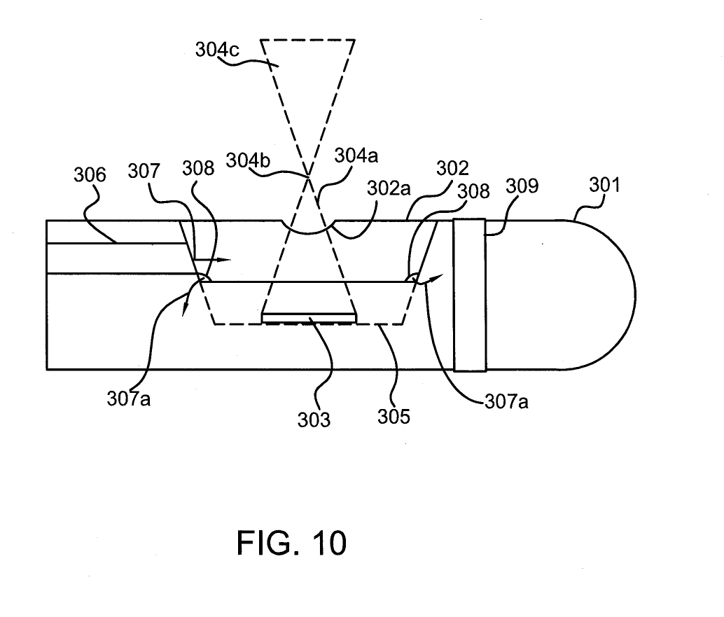

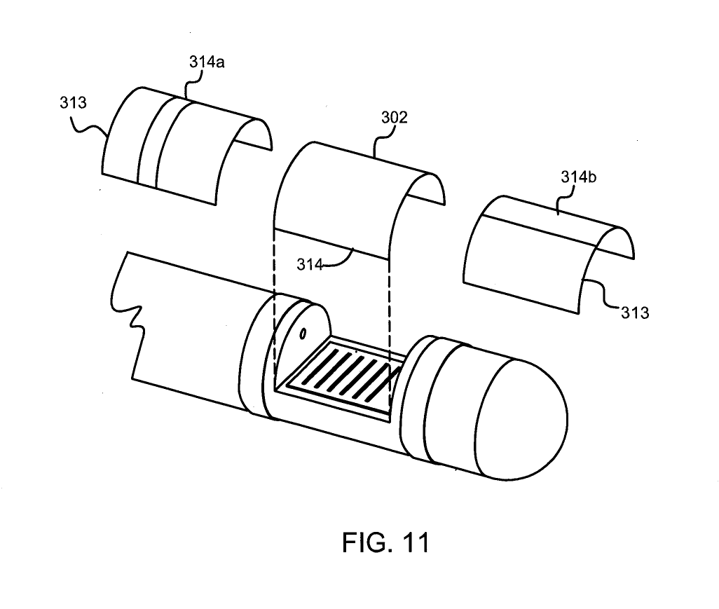

FIG. 10 is a side view of a catheter schematically illustrating an ultrasound compatible ablation member for a catheter with a premounted side viewing ultrasound array. The catheter 301 has an ultrasound compatible ablation member 302 covering the interior region 305 containing a premounted side viewing ultrasound array 303. FIG. 10 shows a transparent side view of the catheter 301 with the ultrasound compatible ablation member 302 mounted in a manner which permits ablations on the side of the catheter while using an ultrasound transducer 303 and one or more mapping electrodes 309. The ablation member 302 is curved to permit a smooth catheter profile, but may take any number of shapes and surface features such as an arcing surface with flat facets, such as those on the surface of a faceted diamond.

The array 303 can be located anywhere in the interior volume 305, but may preferentially be located below the center line (longitudinal axis) of the catheter to permit the ultrasound beam 304a to focus at a point 304b which is close to the catheter. This location of the array 303 well below the center line helps to avoid the undesirable coherently added echo reflections from the inside surface of the ultrasound compatible ablation member 302. A hole 302a in the ultrasound compatible ablation member 302 may be made to allow for a great proportion of the acoustic energy to be transmitted through this hole with very little degradation in the effectiveness of the metal electrode on the ablation member 302 as an EP ablation electrode.

The irrigation fluid path escape holes may be placed at any number of positions, but the position with the best effectiveness may be a hole 302a as shown. As discussed earlier, the irrigation fluid inflow 307 is brought to the interior volume or chamber 305 by a lumen 306. The walls of this chamber 305 are angled as shown to avoid undesired ultrasound echoes from the walls of this chamber. Similarly as described with the ultrasound compatible ablation member mounted at the tip of the catheter, the shape of the surface of the ultrasound compatible ablation member 302 may be either smooth or in plates or faceted to permit good ultrasound performance (i.e., good echo transmission through the ablation member but with few coherent echoes from the surface thereof). Since this chamber 305 for the ultrasound array 303 is located near the tip of the catheter, there should be plenty of room for the steering wire assemblies needed to steer the catheter. These anchor points can be made in the catheter region to the “left” of the chamber 305 region.

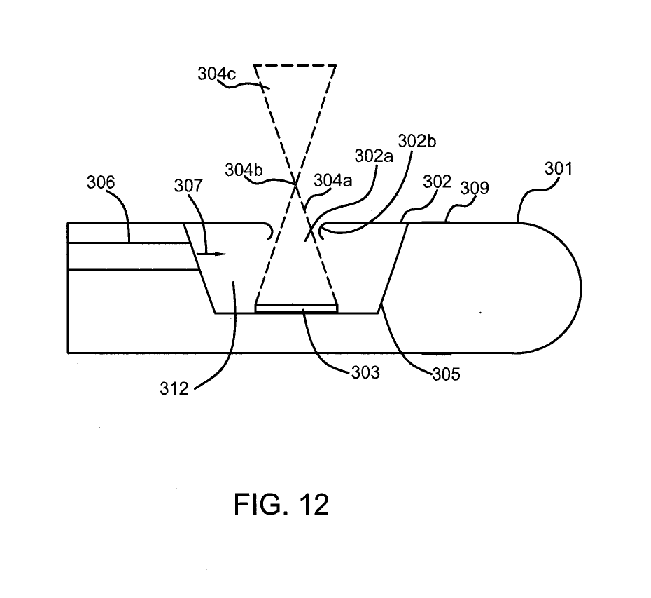

An interior region (312 in FIG. 12) exists underneath the ultrasound compatible ablation member 302 which contains an ultrasound medium such as saline. The medium may be made of a non-moving material, but preferably the medium is saline and supplied as a flow of saline 307 through the lumen 306 in the catheter 301. The ablation member 302 will preferably possess one or more holes in the surface. These openings in the ablation member 302 allow for saline flow out 307a of the catheter in gaps 308 at the “corners” of the ablation member or through holes 302a which may number as a single hole as shown, or multiple holes. The flow of saline in this way permits the following very important operational features including, for example, device safety, as no pre-loaded material needs to be placed under the UCRAE prior to use, ablation cooling, and removal of undesired small air bubbles which can be trapped during the initial filling. Initial filling and adequate flushing will be done prior to use in a manner which is common practice as with mechanical IVUS catheters.

FIG. 11 is a perspective view illustrating different ultrasound compatible ablation members. Three examples of the ablation member 302 are shown. The ablation member 302 includes an electrode support layer 313 and a metal electrode 314. The metal electrode 314 itself can be patterned, as shown in the examples 314a and 314b; many other patterns are possible. No holes 302a are shown here, only for brevity.

FIG. 12 is a side sectional view of the catheter of FIG. 10, showing the catheter 301 with the ultrasound compatible ablation member 302 covering an interior region 312 containing the premounted side viewing ultrasound transducer device 303 to form the “side viewing” device. The ultrasound transducer 303 can be a single element or an array of elements. The ultrasound transducer, preferably an array of elements, can be located anywhere in the interior volume 312, but may preferentially be located below the center line of the catheter to permit the ultrasound beam 304a to focus at a near field point 304b in the tissue during ablation.

The saline escape hole(s) 302a in the ultrasound compatible ablation member 302 may be made to allow for a great proportion of the acoustic energy to be transmitted through this hole with very little degradation in the effectiveness of the ablation member metal electrode as an EP ablation electrode. The water path escape holes may be placed at any number of positions, but the position with the best effectiveness is likely a hole 302a as shown. The hole(s) 302a in the ablation member 302 may be made with a rounded-rim feature 302b to enhance safety by assuring an atraumatic ablation member surface. The walls of this chamber 305 are preferably angled as shown to avoid undesired ultrasound echoes from the walls of this chamber.

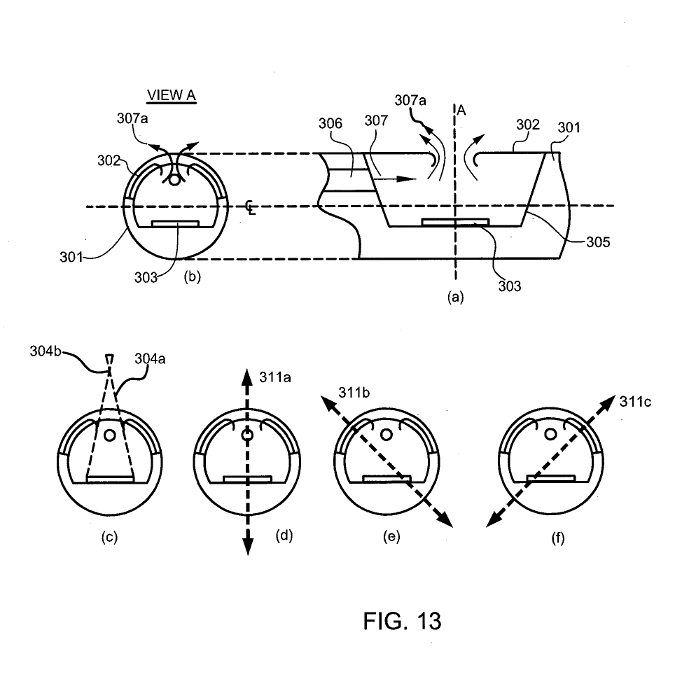

FIG. 13 shows a set of sectional views of the side viewing catheter of FIG. 12 illustrating different bi-directional steering configurations. The hole(s) 302a on the surface of the ultrasound compatible ablation member may be either round or oval in shape. FIG. 13a shows a front sectional view; FIG. 13b shows a side sectional view illustrating the saline flow 307a; and FIG. 13c shows a side sectional view illustrating the ultrasound beam 304a to focus at the point 304b. The axis of catheter bi-directional steering may be arranged to be along the axis 311a in FIG. 13d, the axis 311b in FIG. 13e, or the axis 311c in FIG. 13f, in accordance with the anatomy for the device to be used. In general, the bi-directional steering along the axis 311a will be preferred in order to align the ablation surface with the tissue to be ablated. The ability for the operator to visualize the tissue surface, and subsequently gain ablation feedback information, is valuable. Ultrasound feedback information may indicate when a sufficient ablation is achieved, and guide the procedure through RF power titration to avoid several undesirable ablation problems such as coagulum formation, tissue wall perforation, and vessel ostia stenosis.

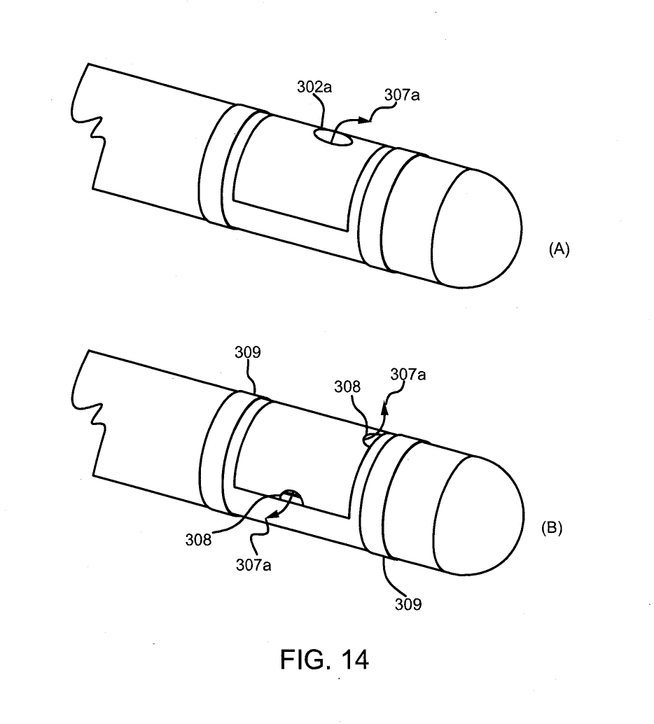

FIGS. 14a and 14b show different arrangements of hole shapes and positions of the holes 302a and gaps 308 in the ultrasound compatible ablation member 302 to permit irrigation fluid flow 307a. These variants shown are not mutually exclusive; many variations with hole positions, shapes, and numbers may be used to tailor a specific design requirement.

In the description, numerous details are set forth for purposes of explanation in order to produce a thorough understanding of the present invention. However, it will be apparent to one skilled in the art that not all of these specific details are required in order to practice the present invention. Additionally, while specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. An ultrasound compatible ablation catheter, comprising:
a catheter body having a distal end and an ultrasonic transducer directing ultrasonic beams for imaging a target; and
an ablation electrode connected to the catheter body, the ablation electrode having a plastic shell and a metallic coating on the plastic shell which are disposed in a path of the ultrasonic beams of the ultrasonic transducer between the ultrasonic transducer and the target, the metallic coating of the ablation electrode to be energized for ablation.

2. The catheter of claim 1,
wherein the plastic shell has an acoustic impedance magnitude which is in a range of 1500×10³ to 1750×10³ Rayls (kg/m²s) at a temperature of 37° C.

3. The catheter of claim 1,
wherein the metallic coating is substantially thinner than the plastic shell.

4. The catheter of claim 3,
wherein the thickness of the plastic shell is at least about 10 times the thickness of the metallic coating.

5. The catheter of claim 3,
wherein the plastic shell has a thickness of at most about 500 microns and the metallic coating has a thickness of at most about 20 microns.

6. The catheter of claim 1,
wherein the plastic shell comprises TPX® (polymethylpentene).

7. The catheter of claim 1,
wherein the ablation region further comprises an electrical barrier layer on an exterior surface of the metallic coating, the barrier layer being substantially thinner than the metallic coating.

8. The catheter of claim 1,
wherein the catheter body and the ablation electrode form a fluid cavity to store a fluid through which the ultrasonic beams of the ultrasonic transducer are transmitted across the ablation electrode to the target.

9. The catheter of claim 8,
wherein the plastic shell has an acoustic impedance which is substantially equal to an acoustic impedance of the fluid.

10. The catheter of claim 8, further comprising:
at least one fluid entry port for the fluid cavity, and at least one fluid exit port for the fluid cavity.

11. The catheter of claim 1,
wherein the ablation electrode comprises an ablation tip disposed near the distal end.

12. The catheter of claim 11,
wherein the ablation tip is dome-shaped to provide a rounded ablation surface on the metallic coating.

13. The catheter of claim 1,
wherein the ablation electrode has one of an uneven surface or a faceted surface to scatter reflective energy of the ultrasonic beams passing therethrough between the ultrasonic transducer and the target.

14. The catheter of claim 1,
wherein the ultrasonic transducer is disposed on the distal end of the catheter body and comprises an array for forward looking imaging.

15. The catheter of claim 1,
wherein the ultrasonic transducer and the ablation electrode are disposed on the catheter body, and the ultrasonic transducer comprises an array for side looking imaging.

16. The catheter of claim 15,
wherein the ultrasonic transducer and the ablation electrode are disposed on opposite sides with respect to a longitudinal axis of the catheter body.

17. The catheter of claim 1, further comprising:
a control unit which controls an ultrasound generator to supply ultrasound energy to the ultrasonic transducer, an ultrasound receiver to accept echo signals, and an RF energy source to supply RF energy to the metallic coating of the ablation electrode, for ultrasound imaging and RF ablation simultaneously.

18. The catheter of claim 1,
wherein the ablation electrode is constructed of materials and thicknesses to produce an absorption loss of less than about 50% of ultrasonic beam energy of the ultrasonic beams of the ultrasonic transducer for imaging the target.

19. An ultrasound compatible ablation catheter, comprising:
a catheter body having a distal end and an ultrasonic transducer directing ultrasonic beams for imaging a target; and
an ablation electrode connected to the catheter body, the ablation electrode having a plastic shell and a metallic coating on the plastic shell which are disposed in a path of the ultrasonic beams of the ultrasonic transducer between the ultrasonic transducer and the target, the metallic coating of the ablation electrode to be energized for ablation;
wherein the catheter body and the ablation electrode form a fluid cavity to contain a fluid through which the ultrasonic beams of the ultrasonic transducer are transmitted across the ablation electrode to the target;
wherein the plastic shell has an acoustic impedance magnitude which is in a range of 1500×10³ to 1750×10³ Rayls (kg/m²s) at a temperature of 37° C.; and
wherein the metallic coating is substantially thinner than the plastic shell.

20. The catheter of claim 19, further comprising:
at least one fluid entry port for the fluid cavity, and at least one fluid exit port for the fluid cavity.

21. The catheter of claim 19,
wherein the ablation tip is constructed of materials and thicknesses to produce an absorption loss of less than about 50% of ultrasonic beam energy of the ultrasonic beams of the ultrasonic transducer for imaging the target.

22. An ultrasound compatible ablation catheter, comprising:
a catheter body having a distal end and an ultrasonic transducer directing ultrasonic beams for imaging a target; and
an ablation electrode connected to the catheter body, the ablation electrode having a plastic shell and a metallic coating on the plastic shell which are disposed in a path of the ultrasonic beams of the ultrasonic transducer between the ultrasonic transducer and the target, the metallic coating of the ablation electrode to be energized for ablation;
wherein the catheter body and the ablation electrode form a fluid cavity to contain a fluid through which the ultrasonic beams of the ultrasonic transducer are transmitted across the ablation electrode to the target;
wherein the plastic shell has an acoustic impedance which is substantially equal to an acoustic impedance of the fluid; and
wherein the metallic coating is substantially thinner than the plastic shell.

23. The catheter of claim 22, further comprising:
at least one fluid entry port for the fluid cavity, and at least one fluid exit port for the fluid cavity.

24. The catheter of claim 22,
wherein the ablation electrode is constructed of materials and thicknesses to produce an absorption loss of less than about 50% of ultrasonic beam energy of the ultrasonic beams of the ultrasonic transducer for imaging the target.
